(19) United States
(12) Patent Application Publication
(10) Pub. No.: US 2026/0174470 A1
(43) Pub. Date: Jun. 25, 2026

(54) CONNECTING ELEMENT FOR CONNECTING MATERIAL

(71) Applicant: CAMP S.P.A., Premana (IT)

(72) Inventors: Antonio Codega, Premana (IT); Andrea Donadoni, Premana (IT)

(73) Assignee: CAMP S.P.A., Premana, LC (IT)

(21) Appl. No.: 18/729,147

(22) PCT Filed: Feb. 6, 2024
(86) PCT No.: PCT/IB2024/051081

§ 371 Date: Jul. 15, 2024

(30) Foreign Application Priority Data
Feb. 6, 2023 (IT) 102023000001833

(51) Int. Cl. A61B 17/70

(52) U.S. Cl. CPC A61B 17/7032

(57) ABSTRACT

The present invention describes a connecting element for connecting at least a first material having a tubular shape delimited by a side wall extending longitudinally from a first element end to a second element end, the first element end delimiting a first end opening, the second element end delimiting a second end opening, a proximal element portion from said first end, a distal element portion from said second element end and a central element portion being defined along the longitudinal extension of the connecting element, wherein the connecting element comprises a first opening in the side wall, and the second element end is inserted through the first opening of the connecting element taking a folded state in which:
the central element portion forms a first material connection loop to which the first material is connectable;
the distal element portion is inside the proximal element portion;
the distal element portion is secured in a fixed position with respect to the proximal element portion.

### FIELD OF THE INVENTION

The present invention relates to a connecting element for connecting material to a rope end.

### TECHNOLOGICAL BACKGROUND

In various applications, such as at height works like rope access, arboriculture, works in confined environments or on pylons and industrial structures, as well as in sports such as mountaineering and climbing, it is common practice to use ropes with a material connection loop at the end.

Over time, several solutions have been developed to form a loop at the end of a rope.

A first known solution consists in folding the rope end backwards so as to form a loop close to the end of the rope. The rope end is positioned parallel to the part of rope that leads to the loop and connected thereto by sewing.

Examples of solutions of this type are described in documents EP 768.100 and EP 1.659.209.

This type of solution is very widespread due to its quick and easy implementation, but it has limitations in relation to the resulting final volume of the rope end, in particular due to the abrupt variation of the section due to the presence of the rope end. In particular, in uses where it is necessary to recover the rope from a distance, the rope end, remaining outside the part of the rope leading to the loop, is susceptible to becoming entangled in narrow passages or equipment such as quickdraws through which the rope runs, making the rope recovery operation difficult and cumbersome.

A further known technique for creating one or more coupling points at the rope end is the technique commonly referred to as eyelet splicing, in which the rope end is folded onto itself to form a loop and in which the strands of the rope end are intertwined with the strands of the part of rope leading to the loop. There are various splicing techniques depending on the type of braiding of the rope and the presence of a core inside the rope.

A splicing solution is described in document JP03479327B9.

Although these solutions do not require the use of sewing and partially solve the above-mentioned volume problems, they however require complex manufacturing techniques. Moreover, the rope end made with the splicing technique is however thicker than the nominal diameter of the rope, hence not only being bulkier, but also more rigid.

A last known solution is to use a flat webbing to create a loop on the rope end. The flat webbing is folded onto itself so as to form the loop. The rope end is then positioned between the two folded edges of the webbing. By sewing, the edges of the flat webbing are firmly connected to the rope end, so as to ensure the correct resistance load.

A solution of this type is described in document DE 102017108623.

Although the possibility of the rope end getting stuck is reduced in solutions of this type, the edges of the flat webbing are sharp and considerably stiffen the rope end, which can still get stuck during rope recovery operations.

A further solution is described in document FR 2.893.378 B1 (PENARD DAVID [FR]), which describes the preparation of “textile shackles” (loop shackle) for maritime application obtained with lines made by weaving and without sewing.

Therefore, the need to have an alternative technical solution to the known solutions described above is strongly felt.

### SUMMARY OF THE INVENTION

It is the object of the present invention to provide a solution allowing the connection of a material which allows avoiding at least partially the drawbacks complained of above with reference to the prior art, and in particular which allows having a material connection loop with reduced volume and thus less susceptible to getting stuck.

It is a further object of the present invention to provide a solution for the connection of a material with low costs and easy to manufacture.

Such an object is achieved by an element according to claim 1 and the dependent claims.

Tit is an object of the present invention also a connection assembly comprising the connecting element and a rope according to claim 17 and the dependent claims.

The present invention also relate to safety devices or parts thereof, for hiking and sports in general, comprising the connecting element described.

Further advantageous embodiments of the connecting element and the connection assembly are the subject of the respective dependent claims.

### BRIEF DESCRIPTION OF THE DRAWINGS

The features and advantages of the present invention will become more apparent from the description of preferred embodiments thereof, given below by way of non-limiting indication, with reference to the accompanying drawings, in which:

FIG. 1 is a perspective view of the connecting element, according to an embodiment of the invention;

FIG. 2 is a section view along the section plane A-A of the connecting element in FIG. 1;

FIG. 3 is a side view of the connecting element in FIG. 1 in the folded state;

FIG. 4 is a section view along the section plane B-B of the connecting element in FIG. 3;

FIG. 5 is a section view along the plane C-C of the connecting element in FIG. 3;

FIG. 6 is a side view of a connection assembly according to an embodiment of the invention;

FIG. 7 is a section view along the plane D-D of the connection assembly in FIG. 6;

FIG. 8 is a section view along the plane D-D of the connection assembly in FIG. 6 in a preferred embodiment;

FIG. 9 is a section view along the section plane A-A of the connecting element, according to an embodiment of the invention;

FIG. 10 shows an example of application of the connecting element of the invention; and

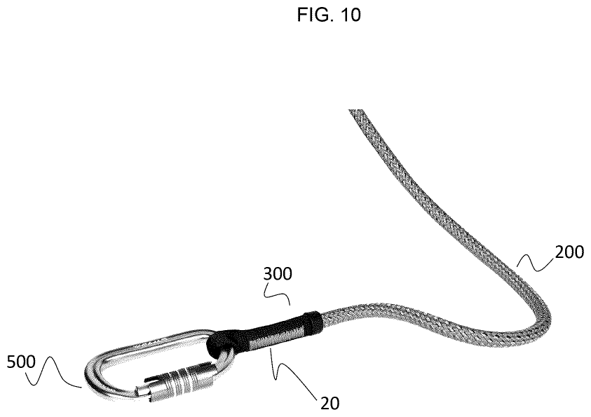

FIG. 11 shows a further embodiment of the present invention.

It should be noted that equal or similar elements in the drawings will be indicated by the same numeric or alphanumeric numerals.

### DETAILED DESCRIPTION OF THE INVENTION

With reference to the aforesaid figures, a connecting element for connecting material is now described, simply referred to as “connecting element” or just “element” below and indicated by reference numeral 100.

For the purposes of the present invention, the term “material” is to be understood in the most general form as any object that, in carrying out the work, can need to be connected to a loop, for example a connector, a karabiner, an anchoring device, etc.

The term “material” is also to be understood as any anchoring element to which the connecting element can be anchored, for example a ring, a karabiner, a link, a beam, a rope, a cable.

For example, such an anchoring can be carried out directly, e.g., by a knot (e.g., by the knot known as “wolf's mouth”) or indirectly through a further connection device.

The connecting element 100 has a tubular shape, since it is at least partially hollow inside.

In particular, the internal cavity is preferably longitudinal.

According to an embodiment, said element is made of a material selected from the group comprising: polyester or polyamide.

According to an embodiment, the connecting element 100 is made of textile material and/or by weaving.

According to an embodiment, the connecting element 100 is represented by a tubular sock or a tubular webbing.

It is possible to define a longitudinal direction L of the connecting element 100 and a radial direction r perpendicular to the longitudinal direction L.

The connecting element 100 has a side wall 1 and extends longitudinally from a first element end 2 to a second element end 3, where said first element end 2 delimits a first opening 4 and said second element end 3 delimits a second opening 5.

It is possible to define, along the longitudinal extension L, a proximal element portion 6 at the first end 2, a distal element portion 7 at the second element end 3 and a central element portion 8 therebetween.

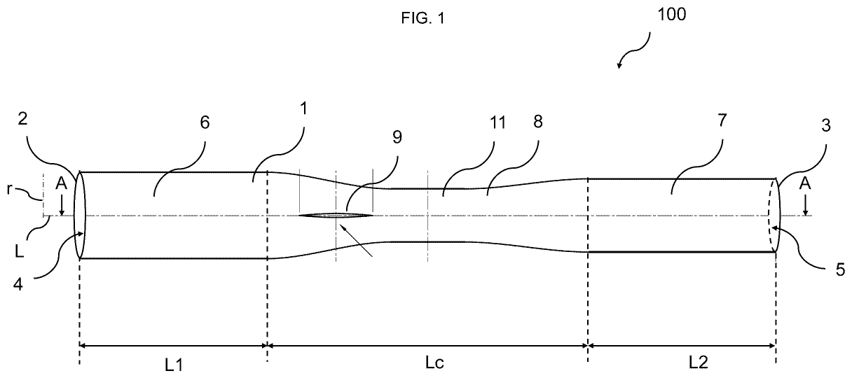

In FIG. 1, the proximal element portion 6, the distal element portion 7 and central element portion 8 are identified by the respective lengths L1, L2 and Lc.

Since they are portions of the connecting element 100, the proximal element portion 6, the distal element portion 7 and the central element portion 8 also have a tubular shape and are at least partially hollow inside.

In the figures, the lengths L1, L2 and Lc should not be understood as indicating specific dimensional ratios, but are used for the sole purpose of identifying each portion of the connecting element 100.

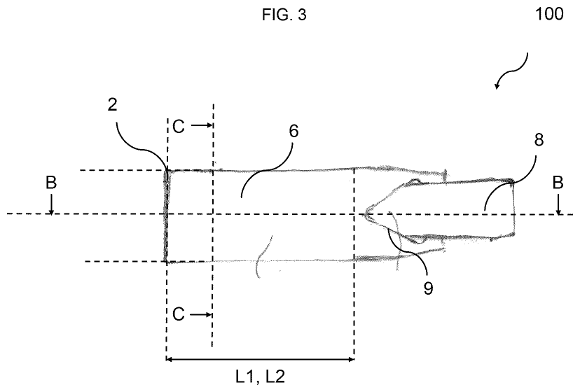

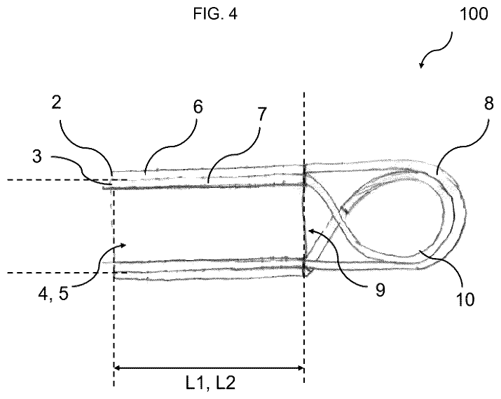

Therefore, although FIGS. 3 and 4 show an exemplary embodiment in which the length L1 of said proximal element portion 6 and the length L2 of said distal element portion 7 are substantially equal, according to an embodiment (not shown in the figures), the length L1 of said proximal element portion 6 and the length L2 of said distal element portion 7 can have a different length.

According to an embodiment, the connecting element 100 has a constant internal section or, according to an alternative embodiment, can comprise one or more portions with a reduced section.

For example, the section can narrow from the proximal element portion 6 or from the distal element portion 7 towards the central element portion 8 or have one or more portions with a narrower section only in the central element portion 8.

According to an aspect of the invention, said connecting element 100 comprises a first opening 9 in the side wall 1.

In an embodiment shown in the figures, said first opening 9 is an opening formed in the side wall 1, e.g., in the central portion of the element 8 closest to the proximal element portion 6.

According to an embodiment, said first opening 9 extends longitudinally into the side wall 1.

According to an embodiment, the first opening 9 is made during the process of manufacturing the connecting element 100; in a preferred embodiment, in fact, said first opening is not made by cutting or perforating the side wall 1.

For example, the opening 9 can be made during the production of the connecting element by weaving; advantageously, this allows the yarn not to be cut.

According to an embodiment not shown in the figures, said first opening 9 can be reinforced along its edge; for example, in the case of manufacturing the connecting element 100 by weaving, it is possible to provide a reinforcement hem of said first opening 9.

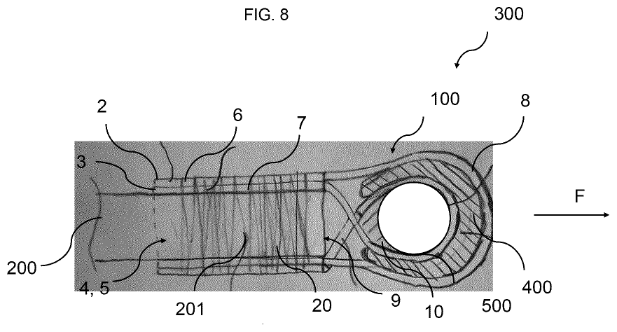

According to an embodiment of the invention, for example shown in FIG. 8, said connecting element 100 can comprise a stiffening 400.

Preferably, said stiffening 400 is at the central element portion 8.

As explained herein below, advantageously, the stiffening 400 allows keeping the material connection loop 10 open, preventing the central element portion 8 in the folded state from being crushed.

Advantageously, the stiffening 400 gives increased load resistance to the connecting element 100.

According to an aspect of the present invention, said stiffening 400 is made by inserting or applying a stiffening element.

In an embodiment, it is possible to provide retention means adapted to keep the stiffening element 400 in position at the central element portion 8.

According to an embodiment, it is possible to provide a pocket in the side wall 1 at the central element portion 8 in which the stiffening element 400 can be housed.

According to a preferred embodiment, the stiffening element 400 is sewn directly onto the side wall 1.

Advantageously, this prevents the stiffening element 400 from slipping out of the connecting element 100 when using the connecting element 100.

It should be noted that the stiffening element 400 is not necessarily an additional and separate element.

According to an embodiment, the stiffening element 400 can be represented by a stiffening or thickening of the side wall 1 at the central element portion 8.

According to the previous embodiment, the stiffening element 400 can thus be made during the manufacturing of the connecting element 100 itself.

FIG. 3 shows the connecting element 100 of the invention in the folded state.

In particular, the connecting element 100 is folded onto itself and the second element end 3 is inserted into the first opening 9 so as to form a material connection loop 10 to which a material 500 or another connection device, such as a karabiner, for example, is connectable.

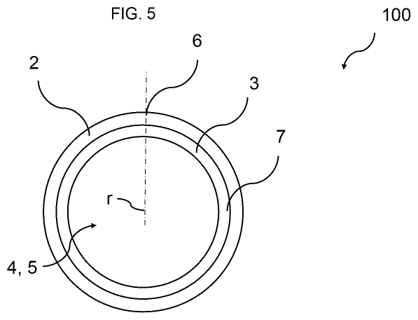

In FIGS. 4 and 5, the distal element portion 7 is internal and can possibly be coaxial to the proximal element portion 6 so that said portions are at least partially superimposed along the radial direction r.

As described later, in the folded state, said connecting element 100 is connectable to a rope end 201 inserted through said second opening 5 into the distal element portion 7.

In the embodiment shown in FIG. 8, the stiffening element 400 allows keeping the material connection loop 10 open.

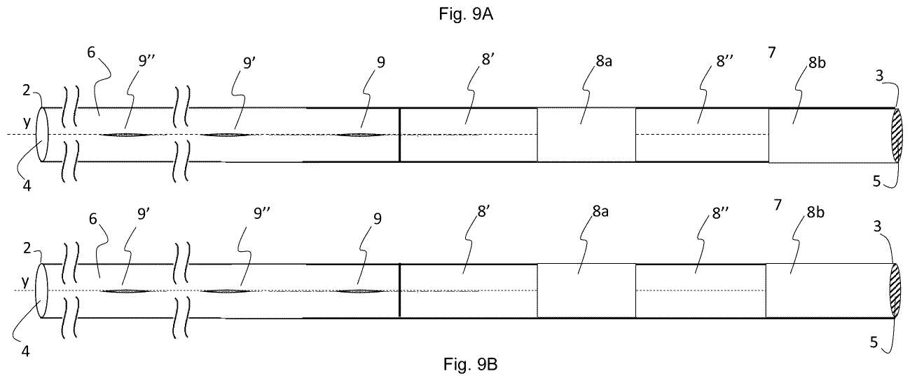

According to a further embodiment of the present invention shown in FIG. 9, the connecting element 100 comprises a second opening 9′ in the side wall 1.

Preferably, said second opening 9′ in the side wall 1 is in a more proximal position as compared to the first opening 9.

Also with reference to the second opening 9′, the above applies in particular with reference to the shape, the production method, and the possible reinforcement.

According to a further embodiment of the present invention also shown in FIG. 9, the connecting element 100 comprises a third opening 9″ in the side wall 1.

According to an aspect of the invention, said third opening 9″ in the side wall 1 is in a more proximal position as compared to the second opening 9′ (FIG. 9A).

According to another aspect of the invention, said third opening 9″ in the side wall 1 is in a more distal position as compared to said second opening 9′ (FIG. 9B).

Also with reference to the third opening 9″, the above applies in particular with reference to the shape, the production method and the possible reinforcement.

Moreover, for the purposes of the present invention, said first opening 9, second opening 9′ and third opening 9″ can be or cannot be substantially on the same longitudinal opening axis y.

As shown in FIGS. 9A and 9B, the connecting element 100 thus has a side wall 1 and extends longitudinally from a first element end 2 to a second element end 3, where said first element end 2 delimits a first opening 4 and said second element end 3 delimits a second opening 5.

Along the longitudinal extension L, there are defined a proximal element portion 6 at the first end 2, a distal element portion 7 at the second element end 3 and a central element portion 8 therebetween.

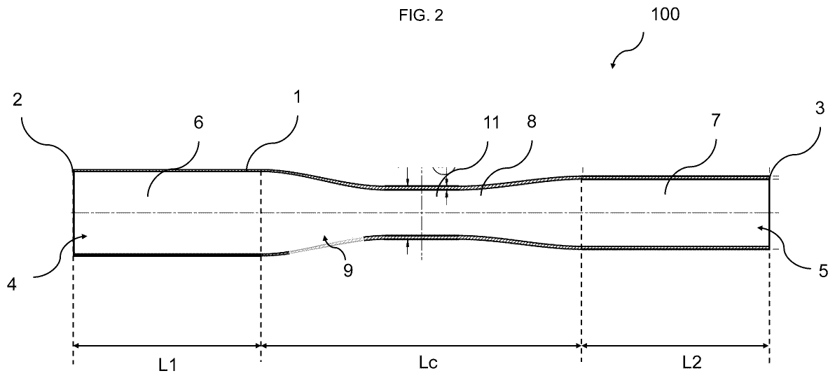

As shown above in relation to FIG. 2, the proximal element portion 6, distal element portion 7 and central element portion 8 are identified by the respective lengths L1, L2 and Lc.

Since they are portions of the connecting element 100, the proximal element portion 6, the distal element portion 7 and the central element portion 8 also have a tubular shape and are at least partially hollow inside.

As shown in FIGS. 9A and 9B, the central element portion 8 comprises a first loop portion 8′, a second insertion portion 8a of the central element portion, a second loop portion 8″ and a second insertion portion 8b of the central element portion.

According to an aspect of the present invention, the first insertion portion 8a and/or the second insertion portion 8b are made in a different color from the first loop portion 8′ and/or the second loop portion 8″.

There is now described a method for connecting a first material 500 to the connecting element 100 of the present invention.

According to an embodiment, in which the connecting element 100 comprises a first opening 9, said connection method comprises the steps of:
1) arranging a connecting element 100 having a first opening 9, as described above in detail;
2) inserting the second end 3 into said first opening 9 creating a first connection loop 10;
3) pulling said second end 3 through the proximal element portion 6, possibly beyond said first end 2, so as to cause the distal element portion 7 to emerge at least partially outside said first end 2;
4) securing said proximal element portion 6 to said distal element portion 7;
5) connecting a first material 500 to the first loop 10 by creating a connection between the first material 500 and the connecting element 100.

According to an embodiment, before step 2) there can be included a step:
2′) of inserting a stiffening element 400 into the central element portion 8.

According to a preferred aspect of the invention, in step 3) the distal element portion 7 is inside (and possibly coaxial to) the proximal element portion 6 so that the distal portion and the proximal portion are at least partially superimposed along the radial direction.

According to a preferred aspect of the invention, step 4) is carried out by sewing 20.

According to a particularly preferred aspect of the invention, step 4) is carried out by a so-called safety sewing.

In particular, said sewing 20 secures the distal element portion 7 in a fixed position with respect to the proximal element portion 6.

A method for connecting a first material 500 and a second material 500′ to the connecting element 100 is described below according to another embodiment of the present invention.

According to this embodiment, the connecting element 100 comprises a central element portion 8 comprising a first loop portion 8′, a second insertion portion 8a of the central element portion, a second loop portion 8″ and a second insertion portion 8b of the central element portion.

In particular, the connecting element 100 further comprises a first opening 9, a second opening 9′ and a third opening 9″, and said connection method comprises the steps of:
1) arranging a connecting element 100 having a first opening 9, a second opening 9′ and a third opening 9″, as described above in detail;
2) inserting the second end 3 of the connecting element 100 into said first opening 9 creating a first loop 10 with a first loop portion of the central element portion 8′;
3) pulling said second end 3 so as to insert the first insertion portion 8a of the central element portion and causing said distal element portion 7 to emerge at least partially from the second opening 9′;
4) possibly securing said distal element portion 7 to said proximal element portion 6;
5) inserting the second end 3 of the connecting element 100 into the third opening 9″ creating a second loop 10′ with said second loop portion 8″ so as to insert the second insertion portion 8b;
6) possibly securing said distal element portion 7 to said proximal element portion;
7) connecting a first material 500 to the first loop 10 and connecting a second material 500′ to said second loop 10′.

According to an embodiment, before step 2) there can be included a step:
2′) of inserting a stiffening 400 into the first loop portion 8′.

According to an embodiment, before step 5) there can be included a step:
5′) of inserting a stiffening 400 into the second loop portion 8″.

According to a preferred aspect of the invention, in step 2) and/or 5) the distal element portion 7 is inside, and possibly coaxial to, the proximal element portion 6 so that they are at least partially superimposed along the radial direction.

According to a preferred aspect of the invention, step 4) is carried out by sewing 20.

According to a particularly preferred aspect of the invention, step 4) is carried out by a so-called safety sewing.

In particular, said sewing 20 secures the first insertion portion 8a in a fixed position with respect to the proximal element portion 6.

According to an embodiment of the invention, step 6) is carried out by sewing.

According to a particularly preferred aspect of the invention, step 6) is carried out by a so-called safety sewing.

In particular, said sewing 20 secures the second insertion portion 8b in a fixed position with respect to the proximal element portion 6.

For the purposes of the present invention, one or both of steps 4) and 6) can be carried out, independently of each other.

According to an alternative aspect of the present invention, in the above-described methods for connecting a first material 500 and possibly a second material 500′ to a connecting element 100, the step of connecting the material 500,500′ can be carried out during the formation of the loop 10,10′ and before the securing step, i.e., between steps 1) and 2) and, 5) and 6), respectively.

Advantageously, this allows the connection of said material 500,500′ in a stable manner.

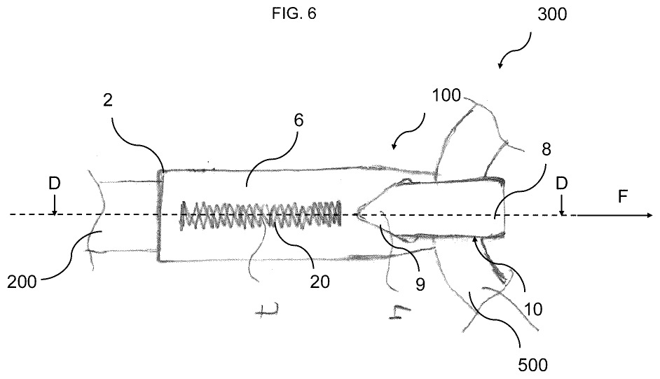

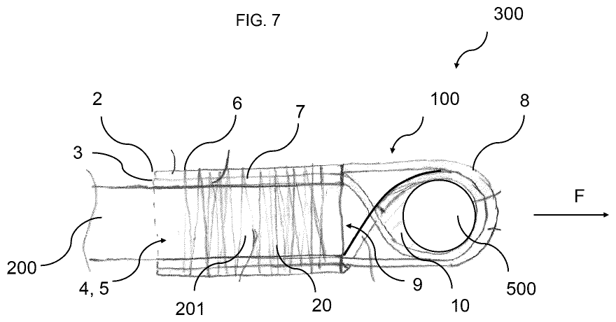

With reference to FIGS. 6, 7, 8, a connection assembly is described, also simply referred to as an assembly below and indicated by reference numeral 300, according to the present invention.

Said connection assembly 300 comprises a connecting element 100, as described above, and a rope 200.

Said rope 200 comprises a rope end 201 at the end thereof.

In said connection assembly 300, the connecting element 100 is in the folded state and said rope end 201 is inserted through said second opening 5 into the distal element portion 7.

In an embodiment, a material 500 is inserted into the material connection loop 10.

In an embodiment, said connecting element 100 is connected to said rope end 201 by sewing 20.

The sewing 20 is preferably a safety sewing or seam sewing.

Said sewing 20 comprises a plurality of stitches passing through the proximal element portion 6, said distal element portion 7, and said rope end 201.

Advantageously, sewing 20 prevents the connecting element 100 from slipping out of the rope end 201, in particular when a tensile load with force F is applied to the connecting element 100, for example through the material 500 inserted into the material connection loop 10.

A method is now described for connecting a material 500 to a rope end 201 according to the present invention.

According to an embodiment, said connecting method comprising the steps of:
1) arranging a connecting element 100, as described above in detail;
2) inserting the second end 3 of the connecting element 100 into said opening 9;
3) pulling said second end 3 through the proximal element portion 6, and beyond said first end 2, so as to cause the distal element portion 7 to emerge at least partially outside said first end 2;
4) inserting said rope end 201 into said distal element portion 7 through said second element opening 5;
5) covering said distal element portion 7 with said proximal element portion 6 so that said distal element portion 7 is inside (and possibly coaxial to) said proximal element portion 6;
6) sewing together said proximal element portion 6, said distal element portion 7, and said rope end 201.

According to an embodiment, before step 2) there can be included a step:
2′) of inserting a stiffening body 400 into the central element portion 8.

According to a preferred aspect of the invention, in step 5) the distal element portion 7 is inside (and possibly coaxial to) the proximal element portion 6 so that the distal portion and the proximal portion are at least partially superimposed along the radial direction.

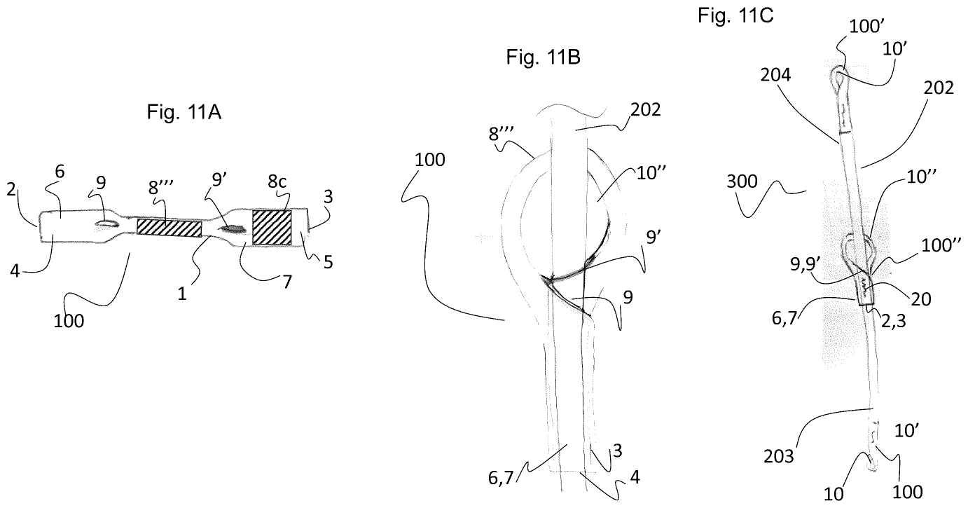

A further embodiment of the present invention is shown in FIG. 11A.

In particular, a connecting element 100 is shown, in which the second opening 9′ in the side wall 1 is in a more distal position as compared to the first opening 9; moreover, it is separated from the first opening 9 by a third loop portion 8″′, whereas in the more distal position as compared to said second opening 9′ there is a third insertion portion 8c.

As shown in FIG. 11B, the second element end 3 can be inserted into the first opening 9 so that the first opening 9 and the second opening 9′ are mutually in communication and matching.

For the purposes of the present invention, said second element end 3 is inserted through the first opening 9 of the side wall of the connecting element 100 taking a folded state in which the third loop portion 8″′ forms a third material connection loop 10″ to which a third material 500″ is connectable.

In particular, in this configuration, the distal element portion 7 can be inside the proximal element portion 6, so that the third insertion portion 8c can be secured in a fixed position with respect to said proximal element portion 6.

In the folded state, said connecting element 100 is connectable to a rope 200 having a rope length 202 having a first end 203 and a second end 204, where said second end 204 is inserted through said first end opening 4 and said second end opening 5, through said first opening 9 and through said second opening 9′, where said third insertion portion 8c is secured in a fixed position with respect to said proximal element portion 6 and is secured in a fixed position with respect to said rope length 202.

For the purposes of the present invention, the securing of the fixed position is obtained by sewing 20, for example by a so-called safety is applied.

The connecting element 100 described above can be used to form the connection assembly 300 shown in FIG. 11C.

In particular, the connection assembly 300 comprises a rope 200 comprising a rope length 202, to the first end 203 of which a first connecting element 100 is connected and to the second end 204 of which a second connecting element 100′ is connected and to which a third connecting element 100″ is connected.

In particular, in the connection assembly 300, the second rope end 204 is inserted through said first end opening 4 and said second end opening 5, through said first opening 9 and through said second opening 9′ of the third connecting element 100″.

In the connection assembly 300 of the present invention, said third connecting element 100″ is secured to the rope length 202 by sewing For the purposes of the present invention, the securing of the fixed position is obtained by sewing 20, for example by a so-called safety is applied.

In the assembly 300 thus obtained, a material 500 can thus be connected not only to the first material connection loop 10 and to the second material connection loop 10′, but also to the third material connection loop 10″.

There is now described a method for connecting a material 500 to a rope 202 having a first end 203 and a second end 204 according to the present invention, regardless of the presence of further connecting elements 100,100′ to said first 203 and/or second 204 ends of the rope.

According to this embodiment, said connecting method comprising the steps of:
1) arranging a connecting element 100″ according to the further embodiment described above;
2) folding said connecting element 100″ forming a third loop 10″ with said third loop portion 8″;
3) inserting said distal element portion 7 into the first opening 9 and pulling the distal portion 7 into the proximal portion 6 until the second opening 9′ meets the first opening 9;
4) inserting the second rope end 204 through the proximal element portion 6 and through the distal element portion 7 and then through said first opening 9 and said second opening 9′;
5) securing said proximal element portion 6 to said distal element portion 7 and to said rope length 202.

According to an embodiment, before step 2) there can be included a step:
2′) of inserting a stiffening body 400 into the third loop portion 8″.

According to a preferred aspect of the invention, in step 5) the distal element portion 7 is inside (and possibly coaxial to) the proximal element portion 6 so that the distal portion and the proximal portion are at least partially superimposed along the radial direction.

The connecting element and the connection assembly of the present invention can form part of a device for a worker's personnel safety, e.g., on sites or in so-called works “at height” or in arboriculture, or devices for the safety of athletes or hikers, or equipment for athletes or hikers (for example, shoulder straps for backpacks) or, again, to carry out recreational activities (playgrounds, adventure parks) or rescue activities.

Such devices include, for example: harness or a part thereof (chest strap, shoulder strap, thigh strap) climbing set, climbing quickdraw.

As can be noted, the object of the invention is fully achieved.

Firstly, as highlighted above, the connecting element 100 allows connecting a material 500 at a rope end 201 ensuring distinctly lower thicknesses and volumes and greater compactness.

The small volume as well as the absence of sharp edges significantly reduces the risk of getting stuck and hooked on other elements of the equipment or the surrounding environment (roofs, slabs, pylons, cables, ladders, plant branches, roughness or protrusions in the ground or rocks) during work, sporting or rescue activities.

Moreover, the preferred tubular shape, materials and manufacturing process by weaving for the connecting element 100 allow providing a connection assembly 300 in which the rope end 201 is substantially unstiffened.

Advantageously, it is possible to connect a plurality of materials in the material connection loop 10.

When provided, the stiffening body 400 allows keeping the material connection loop 10 open, making the use of the device of the invention even simpler.

The connecting element 100 forms a connection assembly 300 with suitable mechanical properties, in particular under the tensile load action, according to safety legislation in force.

Those skilled in the art will be able to make changes or adaptations to the present invention, without however departing from the scope of the following claims.

1. A connecting element for connecting at least a first material having a tubular shape delimited by a side wall extending longitudinally from a first element end to a second element end, said first element end delimiting a first end opening, said second element end delimiting a second end opening, a proximal element portion from said first end, a distal element portion from said second element end and a central element portion being defined along the longitudinal extension of the connecting element,
wherein:
said connecting element comprises a first opening in the side wall,
and said second element end is inserted through said first opening of the connecting element taking a folded state in which:
said central element portion forms a first material connection loop to which said first material is connectable;
said distal element portion is inside said proximal element portion; and
said distal element portion is secured in a fixed position with respect to said proximal element portion.

2. A connecting element according to claim 1, wherein said distal element portion is secured in a fixed position with respect to said proximal element portion by sewing.

3. A connecting element according to claim 1, wherein said first opening is a loop formed in the side wall of the central element portion closer to said proximal element portion.

4. A connecting element according to claim 1, wherein said first opening is longitudinal.

5. A connecting element according to claim 1, wherein the connecting element has a constant internal section or may comprise one or more portions with a reduced section.

6. A connecting element according to claim 1, further comprising one or more stiffening elements at the central element portion.

7. A connecting element according to claim 1, wherein said connecting element is made of textile material or by weaving.

8. A connecting element according to claim 1, wherein said connecting element is a tubular sock or a tubular webbing.

9. A connecting element according to claim 1, further comprising a second opening in the side wall.

10. A connecting element according to claim 1, wherein said second opening in the side wall is in a more proximal position as compared to said first opening.

11. A connecting element according to claim 9, wherein said second element end exits through said second opening.

12. A connecting element according to claim 1, further comprising a third opening in the side wall, a first insertion portion of the central element portion, a second loop portion, and a second insertion portion.

13. A connecting element according to claim 1, wherein said third opening in the side wall is in a more proximal position or a more distal position as compared to said second opening.

14. A connecting element according to claim 12, wherein said second element end is further inserted through said third opening of the side wall of the connecting element taking a folded state in which:
said central element portion forms a first material connection loop to which a first material is connectable,
said first insertion portion is inside the proximal element portion,
said first connection loop portion forms a second material connection loop to which a second material is connectable, and
said first insertion portion is secured in a fixed position with respect to said proximal element portion.

15. A connecting element according to claim 14, wherein said second insertion portion is secured in a fixed position with respect to said proximal element portion.

16. A connecting element according to claim 1, wherein said connecting element is connectable in the folded state to a rope end inserted through said second end opening into the distal element portion.

17. A connection assembly comprising:
a connecting element according to claim 16,
a rope, comprising a rope end;
wherein the connecting element is in the folded state;
wherein said rope end is inserted through said second end opening into the distal element portion.

18. A connection assembly according to claim 17, wherein said connecting element is connected to said rope end by sewing.

19. An assembly according to claim 8, wherein a material connected to the material connection loop.

20. A device comprising the connecting element according to claim 1.

21. A device according to the claim 20, represented by a harness or a part thereof, a backpack, a climbing set, a climbing quickdraw.

22. A connecting element according to claim 1, wherein said second opening in the side wall is in a more distal position as compared to said first opening and separated therefrom by a third loop portion, said connecting element comprising a third insertion portion in a more distal position as compared to said second opening.

23. A connecting element according to claim 1, wherein said second element end is inserted into said first opening so that said first opening is in communication with said second opening.

24. A connecting element according to claim 22, wherein said second element end is inserted through said first opening of the side wall of the connecting element taking a folded state in which:
said third loop portion forms a third material connection loop to which a third material is connectable,
said third insertion portion is inside said proximal element portion and is secured in a fixed position with respect to said proximal element portion.

25. A connecting element according to claim 24, wherein said connecting element is connectable in the folded state to a rope having a rope length, a first end and a second end, where said second end is inserted through said first end opening and said second end opening, through said first opening and through said second opening, where said third insertion portion is secured in a fixed position with respect to said proximal element portion and with respect to said rope length.

26. A connecting element according to claim 25, wherein said fixed position is secured by sewing.

27. A connection assembly comprising:
a connecting element according to claim 22,
a rope having a rope length, a first end and a second end,
wherein the connecting element is in the folded state,
wherein said second end is inserted through said first end opening and said second end opening, through said first opening and through said second opening, where said third insertion portion is secured in a fixed position with respect to said proximal element portion and with respect to said rope length.

28. A connection assembly according to claim 27, wherein said connecting element is secured to said rope length by sewing.

29. An assembly according to claim 28, wherein a material is connected to said third material connection loop.

30. A device comprising the connecting element according to claim 22.
